(19) United States
(12) Patent Application Publication
(10) Pub. No.: US 2011/0287432 A1
(43) Pub. Date: Nov. 24, 2011

(54) SYSTEM AND METHOD FOR TAILORING NUCLEOTIDE CONCENTRATION TO ENZYMATIC EFFICIENCIES IN DNA SEQUENCING TECHNOLOGIES

(75) Inventors: Chiu Tai Andrew Wong, Northford, CT (US); Yi-Ju Chen, New Haven, CT (US); Vinod Bhagwan Makhijani, Guilford, CT (US)

(73) Assignee: 454 Life Science Corporation

(21) Appl. No.: 13/110,589

(22) Filed: May 18, 2011

Related U.S. Application Data
(60) Provisional application No. 61/347,049, filed on May 21, 2010.

(51) Int. Cl. C12Q 1/68; C12M 1/34

(52) U.S. Cl. 435/6.12; 435/287.2

(57) ABSTRACT

An embodiment of a method for optimizing sequencing performance is described that comprises the steps of calculating a nucleotide species specific degradation rate of an apyrase enzyme for a plurality of nucleotide species; determining a concentration for each of the nucleotide species using the nucleotide species specific degradation rate; iteratively providing the concentration of each of the nucleotide species in a reaction environment comprising a polymerase enzyme and a species of template nucleic acid molecule, wherein one or more molecules of the nucleotide species are incorporated into a nascent molecule in a sequencing reaction and the apyrase enzyme is introduced to the reaction environment to degrade unincorporated nucleotide species molecules; and detecting a signal in response to the incorporation of the nucleotide species.

### CROSS-REFERENCE TO RELATED APPLICATIONS

This application is related to and claims priority from U.S. Provisional Patent Application Ser. No. 61/347,049, titled “System and Method for Tailoring Nucleotide Concentration to Enzymatic Efficiencies in DNA Sequencing Technologies”, filed May 21, 2010, which is hereby incorporated by reference herein in its entirety for all purposes.

### FIELD OF THE INVENTION

The invention provides a system and method for determining and using the most advantageous nucleotide concentration in sequencing technologies. More specifically, the invention is based on the detecting the species specific enzymatic efficiencies of apyrase and DNA polymerase as a means of obtaining optimal sequencing performance realized by adjusting concentration of respective nucleotide species based, at least in part, upon the enzymatic efficiencies.

### BACKGROUND OF THE INVENTION

Sequencing-by-synthesis (SBS) generally refers to methods for determining the identity or sequence composition of one or more nucleotides in a nucleic acid sample, wherein the methods comprise the stepwise synthesis of a single strand of polynucleotide molecule complementary to a template nucleic acid molecule whose nucleotide sequence composition is to be determined. For example, SBS techniques typically operate by adding a single nucleic acid (also referred to as a nucleotide) species to a nascent polynucleotide molecule complementary to a nucleic acid species of a template molecule at a corresponding sequence position. The addition of the nucleic acid species to the nascent molecule is generally detected using a variety of methods known in the art that include, but are not limited to what are referred to as pyrosequencing methods that detect light emitted in response to the release of a pyrophosphate molecule, methods that detect a change in pH in response to the release of a Hydrogen ion, or fluorescent detection methods such as those that employ reversible terminators. Typically, the process of adding nucleotide species is iterative until a complete (i.e. all sequence positions are represented) or desired sequence length complementary to the template is synthesized. Some examples of SBS techniques are described in U.S. Pat. Nos. 6,274,320, 7,211,390; 7,244,559; 7,264,929; and 7,335,762 each of which is hereby incorporated by reference herein in its entirety for all purposes.

In some embodiments of SBS, an oligonucleotide primer is designed to anneal to a predetermined, complementary sequence associated with the sample template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species. Alternatively, in some embodiments the primer/template complex is presented with a plurality of nucleotide species of interest (typically A, G, C, and T) at once, and the nucleotide species that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. As described above, incorporation of the nucleotide species can be detected by a variety of methods known in the art, e.g. by detecting the release of pyrophosphate (PPi) (examples described in U.S. Pat. Nos. 6,210,891; 6,258,568; and 6,828,100, each of which is hereby incorporated by reference herein in its entirety for all purposes), by detecting the release of Hydrogen, or via detectable labels bound to the nucleotides. Some examples of detectable labels include but are not limited to mass tags and fluorescent or chemiluminescent labels.

In typical embodiments, unincorporated nucleotides are removed, for example by enzymatic degradation and washing. Enzymatic removal of free nucleotides can be accomplished with the addition of apyrase, especially where washing is inefficient. As those of ordinary skill will appreciate, apyrase works on free nucleotides by breaking the molecular bonds of dNTP molecules producing monophosphate nucleotides and inorganic phosphate (Pi). Thus, dNTPs such as ATP are broken down into dNMP (or AMP in the case of ATP) and 2 Pi molecules. In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Iterations of nucleotide species addition, primer extension, signal acquisition, degradation of excess nucleotide molecules and washing result in a determination of the nucleotide sequence of the template strand.

In typical embodiments of SBS, a large number or population of substantially identical template molecules (e.g. 10³, 10⁴, 10⁵, 10⁶ or 10⁷ molecules) are analyzed simultaneously in any one sequencing reaction, in order to achieve a signal which is strong enough for reliable detection. What is referred to as “homogeneous extension” of nascent molecules associated with substantially all template molecules in a population of a given reaction is required for low signal-to-noise ratios. The term “homogeneous extension”, as used herein, generally refers to the relationship or phase of the extension reaction where each of the substantially identical template molecules described above are homogenously performing the same step in the reaction. For example, each extension reaction associated with the population of template molecules may be described as being in phase or in phasic synchrony with each other when they are performing the same reaction step at the same sequence position for each of the associated template molecules.

Those of ordinary skill in the related art will appreciate that a polymerase extension reaction may result in a small fraction of template molecules in each population to lose or fall out of phasic synchronism with the rest of the template molecules in the population (that is, the reactions associated with the fraction of template molecules either get ahead of, or fall behind, the other template molecules in the sequencing reaction run on the population (some examples are described in Ronaghi, M. Pyrosequencing sheds light on DNA sequencing. Genome Res. 11, 3-11 (2001), which is hereby incorporated by reference herein in its entirety for all purposes). For example, the failure of the polymerase to properly incorporate of one or more nucleotide species into one or more nascent molecules for extension of the sequence by one position results in each subsequent reaction being at a sequence position that is behind and out of phase with the sequence position of the rest of the population. This effect is referred to herein as “incomplete extension” (1E). Alternatively, the improper extension of a nascent molecule by incorporation of one or more nucleotide species in a sequence position that is ahead and out of phase with the sequence position of the rest of the population is referred to herein as “carry forward” (CF). The combined effects of CF and IE are referred to herein as CAFIE. Examples of systems and methods for correction of CAFIE error are described in U.S. patent application Ser. Nos. 12/224,065, titled “System and Method For Correcting Primer Extension Errors in Nucleic Acid Sequence Data”, filed Feb. 15, 2007; and Ser. No. 13/043,063, titled “System and Method to Correct Out of Phase Errors in DNA Sequencing Data by Use of a Recursive Algorithm”, filed Mar. 8, 2011 each of which is hereby incorporated by reference herein in its entirety for all purposes.

With respect to the problem of incomplete extension, there may be several possible mechanisms that contribute to IE that may occur alone or in some combination. One example of a possible mechanism that contributes to IE may include a lack of a nucleotide species being presented to a subset of template/polymerase complexes. Another example of a possible mechanism that contributes to IE may include a failure of a subset of polymerase molecules to incorporate a nucleotide species which is properly presented for incorporation into a nascent molecule. A further example of a possible mechanism that contributes to IE may include the absence of polymerase activity at template/polymerase complexes.

With respect to the problem of CF, there may be several possible mechanisms that contribute to CF that may occur alone or in some combination. For example, one possible mechanism may include excess nucleotide species remaining from a previous cycle. In the present example a result could include a small fraction of an “A” nucleotide species present in a “G” nucleotide species cycle, leading to extension of a small fraction of the nascent molecule if a complementary “T” nucleotide species is present at the corresponding sequence position in the template molecule. Another example of a possible mechanism causing a carry forward effect may include polymerase error, such as the improper incorporation of a nucleotide species into the nascent molecule that is not complementary to the nucleotide species on the template molecule.

Thus, it will be appreciated that in order to minimize the possibility of introducing CAFIE phasic synchrony errors it is highly desirable to present a concentration of nucleotide species in a flow that is optimized to be sufficiently high so that the polymerase can efficiently incorporate the nucleotide species to all members of the population of template molecules during a short period of time while at the same time optimized to be sufficiently low that a degradation enzyme such as apyrase can react with and render the excess nucleotides incapable of incorporation by the polymerase after the incorporation period. It will also be appreciated that the efficiencies of enzymes can be specific to each nucleotide species where an enzyme may be more efficient with one species and less efficient with another.

Therefore, there is a significant advantage in the ability to determine the species specific enzyme efficiencies for both polymerase and apyrase enzyme and employ concentrations of each nucleotide species in sequencing by synthesis reactions that are an optimized balance between incorporation and degradation efficiencies of the enzymes for that species.

### SUMMARY OF THE INVENTION

Embodiments of the invention relate to molecular biology. More particularly, embodiments of the invention relate to methods and systems for tailoring concentrations of each nucleotide species used in sequencing methods based off of the species specific enzymatic efficiencies, particularly the efficiency of apyrase and DNA polymerase to reach the optimal sequencing performance of any given SBS run.

An embodiment of a method for optimizing sequencing performance is described that comprises the steps of calculating a nucleotide species specific degradation rate of an apyrase enzyme for a plurality of nucleotide species; determining a concentration for each of the nucleotide species using the nucleotide species specific degradation rate; iteratively providing the concentration of each of the nucleotide species in a reaction environment comprising a polymerase enzyme and a species of template nucleic acid molecule, wherein one or more molecules of the nucleotide species are incorporated into a nascent molecule in a sequencing reaction and the apyrase enzyme is introduced to the reaction environment to degrade unincorporated nucleotide species molecules; and detecting a signal in response to the incorporation of the nucleotide species.

In addition, an embodiment of a method for optimizing sequencing performance is described that comprises the steps of: introducing a plurality of relative concentrations of nucleotide species into a type of reaction environment comprising a polymerase enzyme, an apyrase enzyme, and a species of a template nucleic acid molecule to produce an uncorrected sequence composition of the species of template nucleic acid molecule; determining a completion efficiency value and a carry forward value for each of the nucleotide species from the uncorrected sequence composition and a reference sequence composition of the species of template nucleic acid molecule; and identifying a species specific concentration for each of a plurality of nucleic acid species using the nucleotide species specific completion efficiency value and the nucleotide species specific carry forward value, wherein the species specific concentrations are optimized to minimize error produced by a sequencing reaction in the type of reaction environment.

In some implementations the method further comprises the steps of: executing the sequencing reaction using the type of reaction environment, wherein the sequencing reaction comprises the steps of: iteratively delivering each of the nucleic acid species at the species specific concentration to the type of reaction environment comprising a second species of template nucleic acid molecule and the polymerase enzyme, wherein the apyrase enzyme is delivered to the reaction environment between iterations of delivery of the nucleic acid species; and detecting a plurality of signals generated in response to incorporation of the nucleic acid species by the polymerase.

Further, an embodiment of a system for optimizing sequencing performance is described that comprises: a computer comprising executable code stored thereon wherein the executable code performs a method comprising the steps of: calculating a nucleotide species specific degradation rate of an apyrase enzyme for a plurality of nucleotide species; and determining a concentration for each of the nucleotide species using the nucleotide species specific degradation rate, wherein the concentration for each of the nucleotide species is determined to provide a balance of the species specific degradation rate relative to an incorporation efficiency of a polymerase enzyme; and a sequencing instrument that performs a method comprising the steps of: iteratively providing the concentration of each of the nucleotide species in a reaction environment comprising a polymerase enzyme and a species of template nucleic acid molecule, wherein one or more molecules of the nucleotide species are incorporated into a nascent molecule in a sequencing reaction and the apyrase enzyme is introduced to the reaction environment to degrade unincorporated nucleotide species molecules; and detecting a signal in response to the incorporation of the nucleotide species.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the FIGURE in which the references element first appears (for example, element 160 appears first in FIG. 1). All of these conventions, however, are intended to be typical or illustrative, rather than limiting.

FIG. 1 is a functional block diagram of one embodiment of a sequencing instrument under computer control and a reaction substrate.

### DETAILED DESCRIPTION OF THE INVENTION

As will be described in greater detail below, embodiments of the presently described invention includes a system and method for tailoring concentration of nucleotide species to the species specific enzymatic efficiencies in sequencing technologies. In particular, embodiments of the invention relate to the optimization of nucleotide concentrations based on the reaction rates of the enzymes of a SBS system, namely apyrase and DNA polymerase.

### a. General

The term “flowgram” generally refers to a graphical representation of sequence data generated by SBS methods, particularly pyrophosphate based sequencing methods (also referred to as “pyrosequencing”) and may be referred to more specifically as a “pyrogram”.

The term “read” or “sequence read” as used herein generally refers to the entire sequence data obtained from a single nucleic acid template molecule or a population of a plurality of substantially identical copies of the template nucleic acid molecule.

The terms “run” or “sequencing run” as used herein generally refer to a series of sequencing reactions performed in a sequencing operation of one or more template nucleic acid molecules.

The term “flow” as used herein generally refers to a single cycle that is typically part of an iterative process of introduction of fluid solution to a reaction environment comprising a template nucleic acid molecule, where the solution may include a nucleotide species for addition to a nascent molecule or other reagent, such as buffers, wash solutions, or enzymes that may be employed in a sequencing process or to reduce carryover or noise effects from previous flows of nucleotide species.

The term “flow cycle” as used herein generally refers to a sequential series of flows where a fluid comprising a nucleotide species is flowed once during the cycle (i.e. a flow cycle may include a sequential addition in the order of T, A, C, G nucleotide species, although other sequence combinations are also considered part of the definition). Typically, the flow cycle is a repeating cycle having the same sequence of flows from cycle to cycle.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

The term “test fragment” or “TF” as used herein generally refers to a nucleic acid element of known sequence composition that may be employed for quality control, calibration, or other related purposes.

The term “primer” as used herein generally refers to an oligonucleotide that acts as a point of initiation of DNA synthesis under conditions in which synthesis of a primer extension product complementary to a nucleic acid strand is induced in an appropriate buffer at a suitable temperature. A primer is preferably a single stranded oligodeoxyribonucleotide.

A “nascent molecule” generally refers to a DNA strand which is being extended by the template-dependent DNA polymerase by incorporation of nucleotide species which are complementary to the corresponding nucleotide species in the template molecule.

The terms “template nucleic acid”, “template molecule”, “target nucleic acid”, or “target molecule” generally refer to a nucleic acid molecule that is the subject of a sequencing reaction from which sequence data or information is generated.

The term “nucleotide species” as used herein generally refers to the identity of a nucleic acid monomer including purines (Adenine, Guanine) and pyrimidines (Cytosine, Uracil, Thymine) typically incorporated into a nascent nucleic acid molecule.

The term “monomer repeat” or “homopolymers” as used herein generally refers to two or more sequence positions comprising the same nucleotide species (i.e. a repeated nucleotide species).

The term “homogeneous extension” as used herein, generally refers to the relationship or phase of an extension reaction where each member of a population of substantially identical template molecules is homogenously performing the same extension step in the reaction.

The term “completion efficiency” as used herein generally refers to the percentage of nascent molecules that are properly extended during a given flow.

The term “incomplete extension rate” as used herein generally refers to the ratio of the number of nascent molecules that fail to be properly extended over the number of all nascent molecules.

The term “genomic library” or “shotgun library” as used herein generally refers to a collection of molecules derived from and/or representing an entire genome (i.e. all regions of a genome) of an organism or individual.

The term “amplicon” as used herein generally refers to selected amplification products, such as those produced from Polymerase Chain Reaction or Ligase Chain Reaction techniques.

The term “variant” or “allele” as used herein generally refers to one of a plurality of species each encoding a similar sequence composition, but with a degree of distinction from each other. The distinction may include any type of variation known to those of ordinary skill in the related art, that include, but are not limited to, polymorphisms such as single nucleotide polymorphisms (SNPs), insertions or deletions (the combination of insertion/deletion events are also referred to as “indels”), differences in the number of repeated sequences (also referred to as tandem repeats), and structural variations.

The term “allele frequency” or “allelic frequency” as used herein generally refers to the proportion of all variants in a population that is comprised of a particular variant.

The term “key sequence” or “key element” as used herein generally refers to a nucleic acid sequence element (typically of about 4 sequence positions, i.e., TGAC or other combination of nucleotide species) associated with a template nucleic acid molecule in a known location (i.e., typically included in a ligated adaptor element) comprising known sequence composition that is employed as a quality control reference for sequence data generated from template molecules. The sequence data passes the quality control if it includes the known sequence composition associated with a Key element in the correct location.

The term “keypass” or “keypass well” as used herein generally refers to the sequencing of a full length nucleic acid test sequence of known sequence composition (i.e., a “test fragment” or “TF” as referred to above) in a reaction well, where the accuracy of the sequence derived from TF sequence and/or Key sequence associated with the TF or in an adaptor associated with a target nucleic acid is compared to the known sequence composition of the TF and/or Key and used to measure of the accuracy of the sequencing and for quality control. In typical embodiments, a proportion of the total number of wells in a sequencing run will be keypass wells which may, in some embodiments, be regionally distributed.

The term “blunt end” as used herein is interpreted consistently with the understanding of one of ordinary skill in the related art, and generally refers to a linear double stranded nucleic acid molecule having an end that terminates with a pair of complementary nucleotide base species, where a pair of blunt ends are typically compatible for ligation to each other.

The term “sticky end” or “overhang” as used herein is interpreted consistently with the understanding of one of ordinary skill in the related art, and generally refers to a linear double stranded nucleic acid molecule having one or more unpaired nucleotide species at the end of one strand of the molecule, where the unpaired nucleotide species may exist on either strand and include a single base position or a plurality of base positions (also sometimes referred to as “cohesive end”).

The term “SPRI” as used herein is interpreted consistently with the understanding of one of ordinary skill in the related art, and generally refers to the patented technology of “Solid Phase Reversible Immobilization” wherein target nucleic acids are selectively precipitated under specific buffer conditions in the presence of beads, where said beads are often carboxylated and paramagnetic. The precipitated target nucleic acids immobilize to said beads and remain bound until removed by an elution buffer according to the operator's needs (DeAngelis, Margaret M. et al: Solid-Phase Reversible Immobilization for the Isolation of PCR Products. Nucleic Acids Res (1995), Vol. 23:22; 4742-4743, which is hereby incorporated by reference herein in its entirety for all purposes).

The term “carboxylated” as used herein is interpreted consistently with the understanding of one of ordinary skill in the related art, and generally refers to the modification of a material, such as a microparticle, by the addition of at least one carboxl group. A carboxyl group is either COOH or COO—.

The term “paramagnetic” as used herein is interpreted consistently with the understanding of one of ordinary skill in the related art, and generally refers to the characteristic of a material wherein said material's magnetism occurs only in the presence of an external, applied magnetic field and does not retain any of the magnetization once the external, applied magnetic field is removed.

The term “bead” or “bead substrate” as used herein generally refers to any type of solid phase particle of any convenient size, of irregular or regular shape and which is fabricated from any number of known materials such as cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (as described, e.g., in Merrifield, Biochemistry 1964, 3, 1385-1390), polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and other solid phase bead supports known to those of skill in the art.

The term “reaction environment” as used herein generally refers to a volume of space in which a reaction can take place typically where reactants are at least temporarily contained or confined allowing for detection of at least one reaction product. Examples of a reaction environment include but are not limited to cuvettes, tubes, bottles, as well as one or more depressions, wells, or chambers on a planar or non-planar substrate.

Some exemplary embodiments of systems and methods associated with sample preparation and processing, generation of sequence data, and analysis of sequence data are generally described below, some or all of which are amenable for use with embodiments of the presently described invention. In particular, the exemplary embodiments of systems and methods for preparation of template nucleic acid molecules, amplification of template molecules, generating target specific amplicons and/or genomic libraries, sequencing methods and instrumentation, and computer systems are described.

In typical embodiments, the nucleic acid molecules derived from an experimental or diagnostic sample should be prepared and processed from its raw form into template molecules amenable for high throughput sequencing. The processing methods may vary from application to application, resulting in template molecules comprising various characteristics. For example, in some embodiments of high throughput sequencing, it is preferable to generate template molecules with a sequence or read length that is at least comparable to the length that a particular sequencing method can accurately produce sequence data for. In the present example, the length may include a range of about 25-30 base pairs, about 50-100 base pairs, about 200-300 base pairs, about 350-500 base pairs, about 500-1000 base pairs, greater than 1000 base pairs, or other length amenable for a particular sequencing application. In some embodiments, nucleic acids from a sample, such as a genomic sample, are fragmented using a number of methods known to those of ordinary skill in the art. In preferred embodiments, methods that randomly fragment (i.e. do not select for specific sequences or regions) nucleic acids and may include what is referred to as nebulization or sonication methods. It will, however, be appreciated that other methods of fragmentation, such as digestion using restriction endonucleases, may be employed for fragmentation purposes. Also in the present example, some processing methods may employ size selection methods known in the art to selectively isolate nucleic acid fragments of the desired length.

Also, it is preferable in some embodiments to associate additional functional elements with each template nucleic acid molecule. The elements may be employed for a variety of functions including, but not limited to, primer sequences for amplification and/or sequencing methods, quality control elements (i.e. such as Key elements or other type of quality control element), unique identifiers (also referred to as a multiplex identifier or “MID”) that encode various associations such as with a sample of origin or patient, or other functional element.

For example, some embodiments of the described invention comprise associating one or more embodiments of an MID element having a known and identifiable sequence composition with a sample, and coupling the embodiments of MID element with template nucleic acid molecules from the associated samples. The MID coupled template nucleic acid molecules from a number of different samples are pooled into a single “Multiplexed” sample or composition that can then be efficiently processed to produce sequence data for each MID coupled template nucleic acid molecule. The sequence data for each template nucleic acid is de-convoluted to identify the sequence composition of coupled MID elements and association with sample of origin identified. In the present example, a multiplexed composition may include representatives from about 384 samples, about 96 samples, about 50 samples, about 20 samples, about 16 samples, about 12 samples, about 10 samples, or other number of samples. Each sample may be associated with a different experimental condition, treatment, species, or individual in a research context. Similarly, each sample may be associated with a different tissue, cell, individual, condition, drug or other treatment in a diagnostic context. Those of ordinary skill in the related art will appreciate that the numbers of samples listed above are for the purposes of example and thus should not be considered limiting.

In preferred embodiments, the sequence composition of each MID element is easily identifiable and resistant to introduced error from sequencing processes. Some embodiments of MID element comprise a unique sequence composition of nucleic acid species that has minimal sequence similarity to a naturally occurring sequence. Alternatively, embodiments of a MID element may include some degree of sequence similarity to naturally occurring sequence.

Also, in preferred embodiments the position of each MID element is known relative to some feature of the template nucleic acid molecule and/or adaptor elements coupled to the template molecule. Having a known position of each MID is useful for finding the MID element in sequence data and interpretation of the MID sequence composition for possible errors and subsequent association with the sample of origin.

For example, some features useful as anchors for positional relationship to MID elements may include, but are not limited to, the length of the template molecule (i.e. the MID element is known to be so many sequence positions from the 5′ or 3′ end), recognizable sequence markers such as a Key element and/or one or more primer elements positioned adjacent to a MID element. In the present example, the Key and primer elements generally comprise a known sequence composition that typically does not vary from sample to sample in the multiplex composition and may be employed as positional references for searching for the MID element. An analysis algorithm implemented by application 135 may be executed on computer 130 to analyze generated sequence data for each MID coupled template to identify the more easily recognizable Key and/or primer elements, and extrapolate from those positions to identify a sequence region presumed to include the sequence of the MID element. Application 135 may then process the sequence composition of the presumed region and possibly some distance away in the flanking regions to positively identify the MID element and its sequence composition.

Some or all of the described functional elements may be combined into adaptor elements that are coupled to nucleotide sequences in certain processing steps. For example, some embodiments may associate priming sequence elements or regions comprising complementary sequence composition to primer sequences employed for amplification and/or sequencing. Further, the same elements may be employed for what may be referred to as “strand selection” and immobilization of nucleic acid molecules to a solid phase substrate. In some embodiments, two sets of priming sequence regions (hereafter referred to as priming sequence A, and priming sequence B) may be employed for strand selection, where only single strands having one copy of priming sequence A and one copy of priming sequence B is selected and included as the prepared sample. In alternative embodiments, design characteristics of the adaptor elements eliminate the need for strand selection. The same priming sequence regions may be employed in methods for amplification and immobilization where, for instance, priming sequence B may be immobilized upon a solid substrate and amplified products are extended therefrom.

Additional examples of sample processing for fragmentation, strand selection, and addition of functional elements and adaptors are described in U.S. patent application Ser. No. 10/767,894, titled “Method for preparing single-stranded DNA libraries”, filed Jan. 28, 2004; U.S. patent application Ser. No. 12/156,242, titled “System and Method for Identification of Individual Samples from a Multiplex Mixture”, filed May 29, 2008; and U.S. patent application Ser. No. 12/380,139, titled “System and Method for Improved Processing of Nucleic Acids for Production of Sequencable Libraries”, filed Feb. 23, 2009, each of which is hereby incorporated by reference herein in its entirety for all purposes.

Various examples of systems and methods for performing amplification of template nucleic acid molecules to generate populations of substantially identical copies are described. It will be apparent to those of ordinary skill that it is desirable in some embodiments of SBS to generate many copies of each nucleic acid element to generate a stronger signal when one or more nucleotide species is incorporated into each nascent molecule associated with a copy of the template molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above) and Polymerase Chain Reaction (PCR) methods, each of the techniques are applicable for use with the presently described invention. One PCR technique that is particularly amenable to high throughput applications include what are referred to as emulsion PCR methods (also referred to as emPCR™ methods).

Typical embodiments of emulsion PCR methods include creating a stable emulsion of two immiscible substances creating aqueous droplets within which reactions may occur. In particular, the aqueous droplets of an emulsion amenable for use in PCR methods may include a first fluid, such as a water based fluid suspended or dispersed as droplets (also referred to as a discontinuous phase) within another fluid, such as a hydrophobic fluid (also referred to as a continuous phase) that typically includes some type of oil. Examples of oil that may be employed include, but are not limited to, mineral oils, silicone based oils, or fluorinated oils.

Further, some emulsion embodiments may employ surfactants that act to stabilize the emulsion, which may be particularly useful for specific processing methods such as PCR. Some embodiments of surfactant may include one or more of a silicone or fluorinated surfactant. For example, one or more non-ionic surfactants may be employed that include, but are not limited to, sorbitan monooleate (also referred to as Span™ 80), polyoxyethylenesorbitsan monooleate (also referred to as Tween™ 80), or in some preferred embodiments, dimethicone copolyol (also referred to as Abil® EM90), polysiloxane, polyalkyl polyether copolymer, polyglycerol esters, poloxamers, and PVP/hexadecane copolymers (also referred to as Unimer U-151), or in more preferred embodiments, a high molecular weight silicone polyether in cyclopentasiloxane (also referred to as DC 5225C available from Dow Corning).

The droplets of an emulsion may also be referred to as compartments, microcapsules, microreactors, microenvironments, or other name commonly used in the related art. The aqueous droplets may range in size depending on the composition of the emulsion components or composition, contents contained therein, and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a solid substrate such as a bead for attachment of template and amplified copies of the template, amplified copies complementary to the template, or combination thereof. Further, the solid substrate may be enabled for attachment of other type of nucleic acids, reagents, labels, or other molecules of interest.

After emulsion breaking and bead recovery, it may also be desirable in typical embodiments to “enrich” for beads having a successfully amplified population of substantially identical copies of a template nucleic acid molecule immobilized thereon. For example, a process for enriching for “DNA positive” beads may include hybridizing a primer species to a region on the free ends of the immobilized amplified copies, typically found in an adaptor sequence, extending the primer using a polymerase mediated extension reaction, and binding the primer to an enrichment substrate such as a magnetic or sepharose bead. A selective condition may be applied to the solution comprising the beads, such as a magnetic field or centrifugation, where the enrichment bead is responsive to the selective condition and is separated from the “DNA negative” beads (i.e. no or few immobilized copies).

Embodiments of an emulsion useful with the presently described invention may include a very high density of droplets or microcapsules enabling the described chemical reactions to be performed in a massively parallel way. Additional examples of emulsions employed for amplification and their uses for sequencing applications are described in U.S. Pat. Nos. 7,638,276; 7,622,280; 7,842,457; and 7,927,797; and U.S. patent application Ser. No. 11/982,095, each of which is hereby incorporated by reference herein in its entirety for all purposes.

Also embodiments sometimes referred to as Ultra-Deep Sequencing, generate target specific amplicons for sequencing may be employed with the presently described invention that include using sets of specific nucleic acid primers to amplify a selected target region or regions from a sample comprising the target nucleic acid. Further, the sample may include a population of nucleic acid molecules that are known or suspected to contain sequence variants comprising sequence composition associated with a research or diagnostic utility where the primers may be employed to amplify and provide insight into the distribution of sequence variants in the sample. For example, a method for identifying a sequence variant by specific amplification and sequencing of multiple alleles in a nucleic acid sample may be performed. The nucleic acid is first subjected to amplification by a pair of PCR primers designed to amplify a region surrounding the region of interest or segment common to the nucleic acid population. Each of the products of the PCR reaction (first amplicons) is subsequently further amplified individually in separate reaction vessels such as an emulsion based vessel described above. The resulting amplicons (referred to herein as second amplicons), each derived from one member of the first population of amplicons, are sequenced and the collection of sequences are used to determine an allelic frequency of one or more variants present. Importantly, the method does not require previous knowledge of the variants present and can typically identify variants present at <1% frequency in the population of nucleic acid molecules.

Some advantages of the described target specific amplification and sequencing methods include a higher level of sensitivity than previously achieved. Further, embodiments that employ high throughput sequencing instrumentation, such as for instance embodiments that employ what is referred to as a PicoTiterPlate® array (also sometimes referred to as a PTP™ plate or array) of wells provided by 454 Life Sciences Corporation, the described methods can be employed to generate sequence composition for over 100,000, over 300,000, over 500,000, or over 1,000,000 nucleic acid regions per run or experiment and may depend, at least in part, on user preferences such as lane configurations enabled by the use of gaskets, etc. Also, the described methods provide a sensitivity of detection of low abundance alleles which may represent 1% or less of the allelic variants. Another advantage of the methods includes generating data comprising the sequence of the analyzed region. Importantly, it is not necessary to have prior knowledge of the sequence of the locus being analyzed.

Additional examples of target specific amplicons for sequencing are described in U.S. patent application Ser. No. 11/104,781, titled “Methods for determining sequence variants using ultra-deep sequencing”, filed Apr. 12, 2005; PCT Patent Application Serial No. US 2008/003424, titled “System and Method for Detection of HIV Drug Resistant Variants”, filed Mar. 14, 2008; and U.S. Pat. No. 7,888,034, titled “System and Method for Detection of HIV Tropism Variants”, filed Jun. 17, 2009, each of which is hereby incorporated by reference herein in its entirety for all purposes.

Further, embodiments of sequencing may include Sanger type techniques, techniques generally referred to as Sequencing by Hybridization (SBH), Sequencing by Ligation (SBL), or Sequencing by Incorporation (SBI) techniques. Further, the sequencing techniques may include what is referred to as polony sequencing techniques; nanopore, waveguide and other single molecule detection techniques; or reversible terminator techniques. As described above, a preferred technique may include Sequencing by Synthesis methods. For example, some SBS embodiments sequence populations of substantially identical copies of a nucleic acid template and typically employ one or more oligonucleotide primers designed to anneal to a predetermined, complementary position of the sample template molecule or one or more adaptors attached to the template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species. Alternatively, in some embodiments the primer/template complex is presented with a plurality of nucleotide species of interest (typically A, G, C, and T) at once, and the nucleotide species that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. It will also be appreciated that the process of adding a nucleotide species to the end of a nascent molecule is substantially the same as that described above for addition to the end of a primer.

As described above, incorporation of the nucleotide species can be detected by a variety of methods known in the art, e.g. by detecting the release of pyrophosphate (PPi) using an enzymatic reaction process to produce light or via detection of pH change (examples described in U.S. Pat. Nos. 6,210,891; 6,258,568; and 6,828,100, each of which is hereby incorporated by reference herein in its entirety for all purposes), or via detectable labels bound to the nucleotides. Some examples of detectable labels include but are not limited to mass tags and fluorescent or chemiluminescent labels. In typical embodiments, unincorporated nucleotides are removed, for example by washing. Further, in some embodiments the unincorporated nucleotides may be subjected to enzymatic degradation such as, for instance, degradation using the apyrase or pyrophosphatase enzymes as described in U.S. patent application Ser. Nos. 12/215,455, titled “System and Method for Adaptive Reagent Control in Nucleic Acid Sequencing”, filed Jun. 27, 2008; and Ser. No. 12/322,284, titled “System and Method for Improved Signal Detection in Nucleic Acid Sequencing”, filed Jan. 29, 2009; each of which is hereby incorporated by reference herein in its entirety for all purposes.

In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Repeated cycles of nucleotide addition, extension, signal acquisition, and washing result in a determination of the nucleotide sequence of the template strand. Continuing with the present example, a large number or population of substantially identical template molecules (e.g. 10³, 10⁴, 10⁵, 10⁶ or 10⁷ molecules) are typically analyzed simultaneously in any one sequencing reaction, in order to achieve a signal which is strong enough for reliable detection.

In addition, it may be advantageous in some embodiments to improve the read length capabilities and qualities of a sequencing process by employing what may be referred to as a “paired-end” sequencing strategy. For example, some embodiments of sequencing method have limitations on the total length of molecule from which a high quality and reliable read may be generated. In other words, the total number of sequence positions for a reliable read length may not exceed 25, 50, 100, or 500 bases depending on the sequencing embodiment employed. A paired-end sequencing strategy extends reliable read length by separately sequencing each end of a molecule (sometimes referred to as a “tag” end) that comprise a fragment of an original template nucleic acid molecule at each end joined in the center by a linker sequence. The original positional relationship of the template fragments is known and thus the data from the sequence reads may be re-combined into a single read having a longer high quality read length. Further examples of paired-end sequencing embodiments are described in U.S. Pat. No. 7,601,499, titled “Paired end sequencing”; and in U.S. patent application Ser. No. 12/322,119, titled “Paired end sequencing”, filed Jan. 28, 2009, each of which is hereby incorporated by reference herein in its entirety for all purposes.

Some examples of SBS apparatus may implement some or all of the methods described above and may include one or more of a detection device such as a charge coupled device (i.e., CCD camera) or confocal type architecture for optical detection, Ion-Sensitive Field Effect Transistor (also referred to as “ISFET”) or Chemical-Sensitive Field Effect Transistor (also referred to as “ChemFET”) for architectures for ion or chemical detection, a microfluidics chamber or flow cell, a reaction substrate, and/or a pump and flow valves. Taking the example of pyrophosphate based sequencing, embodiments of an apparatus may employ a chemiluminescent detection strategy that produces an inherently low level of background noise.

In some embodiments, the reaction substrate for sequencing may include a planar substrate such as a slide type substrate, an ISFET, or waveguide type reaction substrate that in some embodiments may comprise well type structures. Further the reaction substrate may include what is referred to as a PTP™ array available from 454 Life Sciences Corporation, as described above, formed from a fiber optic faceplate that is acid-etched to yield hundreds of thousands or more of very small wells each enabled to hold a population of substantially identical template molecules (i.e., some preferred embodiments comprise about 3.3 million wells on a 70×75 mm PTP™ array at a 35 μm well to well pitch). In some embodiments, each population of substantially identical template molecule may be disposed upon a solid substrate, such as a bead, each of which may be disposed in one of said wells. For example, an apparatus may include a reagent delivery element for providing fluid reagents to the PTP plate holders, as well as a CCD type detection device enabled to collect photons of light emitted from each well on the PTP plate. An example of reaction substrates comprising characteristics for improved signal recognition is described in U.S. Pat. No. 7,682,816, titled “THIN-FILM COATED MICROWELL ARRAYS AND METHODS OF MAKING SAME”, filed Aug. 30, 2005, which is hereby incorporated by reference herein in its entirety for all purposes. Further examples of apparatus and methods for performing SBS type sequencing and pyrophosphate sequencing are described in U.S. Pat. Nos. 7,323,305 and 7,575,865, both of which are incorporated by reference above.

In addition, systems and methods may be employed that automate one or more sample preparation processes, such as the emPCR™ process described above. For example, automated systems may be employed to provide an efficient solution for generating an emulsion for emPCR processing, performing PCR Thermocycling operations, and enriching for successfully prepared populations of nucleic acid molecules for sequencing. Examples of automated sample preparation systems are described in U.S. Pat. No. 7,927,797, titled “Nucleic acid amplification with continuous flow emulsion”, filed Jan. 28, 2005, which is hereby incorporated by reference herein in its entirety for all purposes.

Also, the systems and methods of the presently described embodiments of the invention may include implementation of some design, analysis, or other operation using a computer readable medium stored for execution on a computer system. For example, several embodiments are described in detail below to process detected signals and/or analyze data generated using SBS systems and methods where the processing and analysis embodiments are implementable on computer systems.

An exemplary embodiment of a computer system for use with the presently described invention may include any type of computer platform such as a workstation, a personal computer, a server, or any other present or future computer. It will, however, be appreciated by one of ordinary skill in the art that the aforementioned computer platforms as described herein are specifically configured to perform the specialized operations of the described invention and are not considered general purpose computers. Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provides one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof.

A processor may include a commercially available processor such as a Celeron®, Core™, or Pentium® processor made by Intel Corporation, a SPARC® processor made by Sun Microsystems, an Athlon™, Sempron™, Phenom™, or Opteron™ processor made by AMD corporation, or it may be one of other processors that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows®-type operating system (such as Windows® XP, Windows Vista®, or Windows®_{—}7) from the Microsoft Corporation; the Mac OS X operating system from Apple Computer Corp. (such as Mac OS X v10.6 “Snow Leopard” operating systems); a Unix® or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium, such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also, a computer may include one or more library files, experiment data files, and an internet client stored in system memory. For example, experiment data could include data related to one or more experiments or assays such as detected signal values, or other values associated with one or more SBS experiments or processes. Additionally, an internet client may include an application enabled to accesses a remote service on another computer using a network and may for instance comprise what are generally referred to as “Web Browsers”. In the present example, some commonly employed web browsers include Microsoft® Internet Explorer 8 available from Microsoft Corporation, Mozilla Firefox® 3.6 from the Mozilla Corporation, Safari 4 from Apple Computer Corp., Google Chrome from the Google™ Corporation, or other type of web browser currently known in the art or to be developed in the future. Also, in the same or other embodiments an internet client may include, or could be an element of, specialized software applications enabled to access remote information via a network such as a data processing application for biological applications.

A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

### b. Embodiments of the Presently Described Invention

As described above embodiments of the described invention are directed to improved systems and methods for optimizing concentration of nucleotide species introduced into a reaction environment based on the species specific efficiencies of one or more enzymes employed in DNA sequencing technologies.

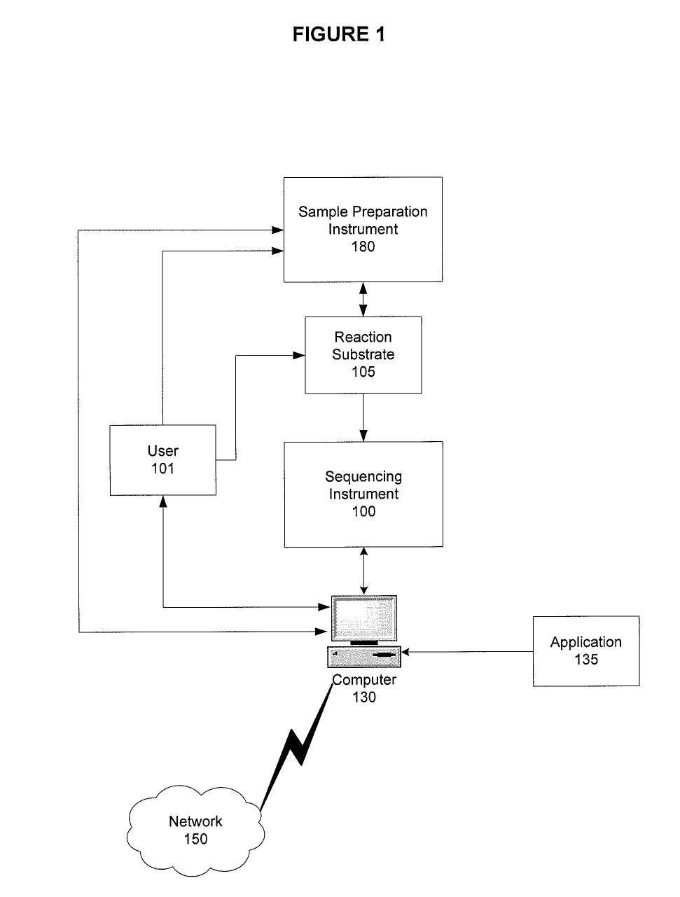

In a typical sequencing embodiment one or more instrument elements may be employed that automate one or more process steps. For example, embodiments of a sequencing method may be executed using instrumentation to automate and carry out some or all process steps. FIG. 1 provides an illustrative example of sequencing instrument 100 that for sequencing processes requiring capture of optical signals typically comprise an optic subsystem and a fluidic subsystem for execution of sequencing reactions and data capture that occur on reaction substrate 105. It will, however, be appreciated that for sequencing processes requiring other modes of data capture (i.e. pH, temperature, electrochemical, etc.) a subsystem for the mode of data capture may be employed which are known to those of ordinary skill in the related art. For instance, a sample of template molecules may be loaded onto reaction substrate 105 by user 101 or some automated embodiment, then sequenced in a massively parallel manner using sequencing instrument 100 to produce sequence data representing the sequence composition of each template molecule. Importantly, user 101 may include any such user that includes but is not limited to an independent researcher, technician, clinician, university, or corporate entity.

Embodiments of sequencing instrument 100 employed to execute sequencing processes may include various fluidic components in the fluidic subsystem, various optical components in an optic subsystem or Field Effect Transistor type detection components (e.g. ISFET or ChemFET) and associated subsystem in the case of pH detection, as well as additional components not illustrated in FIG. 1 but in common use and known to those of ordinary skill in the art that may include microprocessor and/or microcontroller components for local control of some functions. In some embodiments samples may be optionally prepared for sequencing in an automated or partially automated fashion using sample preparation instrument 180 configured to perform some or all of the necessary preparation for sequencing using instrument 100. Further, as illustrated in FIG. 1 sequencing instrument 100 may be operatively linked to one or more external computer components such as computer 130 that may for instance execute system software or firmware such as application 135 that may provide instructional control of one or more of the instruments such as sequencing instrument 100 or sample preparation instrument 180, and/or data analysis functions. Computer 130 may be additionally operatively connected to other computers or servers via network 150 that may enable remote operation of instrument systems and the export of large amounts of data to systems capable of storage and processing. In the present example, sequencing instrument 100 and/or computer 130 may include some or all of the components and characteristics of the embodiments generally described above.

Those of ordinary skill in the related art will appreciate that the performance of a typical sequencing system employing one or more enzymes is sensitive to the efficiency of the various enzymes used in the associated method such as, for instance, the efficiency of polymerase and/or apyrase enzymes used in some sequencing by synthesis methodologies. For example, embodiments of adaptively adjusting the concentration of apyrase in a reaction environment to achieve a desired level of general apyrase activity are described in U.S. patent application Ser. No. 12/215,455, titled “System and Method For Adaptive Reagent Control in Nucleic Acid Sequencing”, filed Jun. 27, 2008, which is hereby incorporated by reference herein in its entirety for all purposes.

It will also be appreciated that the efficiency of any particular enzyme may depend on a number of factors that include, but are not limited to the amino acid sequence composition of an enzyme particularly regions that confer enzyme activity characteristics, and the ambient conditions in a reaction environment. For example, a polymerase may generally comprise a specific activity of about 120,000 units/mg, where “one unit” generally refers to the amount of polymerase enzyme that will incorporate 10 nmol of dNTP's into acid insoluble material in 30 minutes at 65° C. Further, the efficiencies of various polymerase species for incorporating nucleotides, and apyrase species for digesting nucleotides can vary depending upon the species of nucleotide present in the reaction environment.

In some sequencing system embodiments, nucleotide species solutions (dTTP, α-thio-dATP or dATP in some embodiments, dCTP and dGTP) are typically introduced into a reaction environment (i.e. introduction via a “flow” as described above) in a cyclic manner with a flow of apyrase solution in between. In some embodiments, a steady-state concentration of apyrase specific for each nucleotide species is established via the flows of nucleotide species into individual reaction environments on a reaction substrate (i.e. such as the wells of a PicoTiterPlate type substrate described above) during the sequencing process.

The nucleotide species specific steady-state apyrase concentration may be computed using what is referred to as the Michaelis-Menten kinetics to mathematically approximate the kinetic characteristics of an enzyme based upon the assumption that simple kinetic conditions exist. The Michaelis-Menten equation uses constant values that vary by nucleotide species and include the Michaelis constant K_{M} and the maximum rate Vₘₐₓ of enzyme activity in a reaction environment. For example, the constant values for apyrase used in an implementation of a sequencing system are listed in the Table 1 below.

TABLE 1
Enzyme Kinetics of Apyrase Across Multiple Nucleotides

| | dTTP | α-thio-dATP | dCTP | dGTP |
|---|---|---|---|---|
| K_{M} (μM) | 107.2 | 114.8 | 76.5 | 8.1 |
| Vₘₐₓ (μM/10 min) | 294.1 | 232.6 | 250.0 | 47.4 |

In the present example, apyrase is effectively exposed to only one type of nucleotide species at a given time due to the iterative nature of the sequencing reagent flows. The reaction rates between apyrase and each nucleotide species can be estimated using the following formula:

$${V}_{Apy - dNTP} = \frac{{V}_{max} [ dNTP ]}{[ dNTP ] + {K}_{M}} ,$$

where [dNTP] is the concentration of dTTP, dATP, dCTP or dGTP.

Continuing with the present example, the nucleotide species concentrations used for the computation for dTTP, α-thio-dATP, dCTP and dGTP are 40.3, 166.1, 16.3 and 19.7 μM respectively in the reaction environment. It will be appreciated that the concentration value for α-thio-dATP is substantially higher than the concentrations of the other nucleotide species which is due to the modification that has an inhibitory effect upon the incorporation efficiency of a polymerase enzyme (i.e. thus a higher concentration improves the likelihood of polymerase incorporation) as well as an increased processing efficiency of the apyrase as illustrated in greater detail below. In some embodiments the more “natural” dATP species may be used in place of α-thio-dATP and may have a different value for nucleotide species concentration reflecting the higher efficiency of enzyme processing for that species. The estimated activities of apyrase from the Michaelis-Menten calculation towards the different nucleotide species are shown in the Table 2 below. It should again be noted that the calculated efficiency of apyrase to α-thio-dATP illustrated in Table 2 is substantially higher than for the other species and thus apyrase would be expected to process a higher concentration of α-thio-dATP in an efficient manner.

TABLE 2
Estimated Apyrase Activity Across Multiple Nucleotides

| | dTTP | α-thio-dATP | dCTP | dGTP |
|---|---|---|---|---|
| V_{Apy-dNTP} (μM/10 min) | 80.4 | 137.5 | 31.7 | 51.2 |

Table 3 provides a representation of the data from Table 2 in a different way by normalizing the values of concentration and apyrase activity for each of the nucleotide species based upon the value of dCTP because it has the lowest concentration and activity values among the nucleotide species (Nucleotide Species A in Table 3 refers to α-thio-dATP).

TABLE 3
Nucleotide and Apyrase Activities (Normalized)

| Nucleotide | concentration | Apyrase activity |
|---|---|---|
| Species | ratio | ratio |
| T | 2.5 | 2.5 |
| A | 10.2 | 4.3 |
| C | 1.0 | 1.0 |
| G | 1.2 | 1.6 |

In the described embodiments, the concentration of each nucleotide species used in the sequencing process is adjusted based upon the relative level of apyrase activity for that nucleotide species. Those of ordinary skill will appreciate that since apyrase digests nucleotide molecules at a species specific rate, a high apyrase activity for a nucleotide species can reduce the incorporation efficiency of a polymerase enzyme for that species if the concentration of nucleotide molecules available for incorporation is substantially reduced by the apyrase before incorporation can take place. Thus nucleotide concentration for that species has to be higher to counteract the effect of a high species specific apyrase activity.

It will also be appreciated that the estimation of polymerase and apyrase activity using their respective kinetic constants measured outside of a sequencing reaction environment may be different than the levels of activity that actually occur within a reaction environment such as the described reaction wells of a reaction substrate. However, the overall enzymatic efficiency for the combination of apyrase degradation and polymerase incorporation on each nucleotide species can be inferred from raw (i.e. no data correction calculations applied) sequencing data.

For example, Table 4 shows the completion efficiency and carry forward values of individual nucleotide species can be used to optimize the concentrations of each nucleotide species in order to achieve the best sequencing performance. In the present example, DNA Shotgun libraries of the genome C. jejuni were sequenced to calculate the nucleotide species concentrations optimized for polymerase and apyrase efficiencies. The second (TA) and the third (CG) columns of Table 4 show the relative percentage of T and A to C and G nucleotide species concentrations used in sequencing runs where a value for the 100% concentration may be based upon the calculated and normalized species relative ratio values from Table 3. For instance, using the figures from Table 3 100% of the G nucleotide species is 1.2 times greater the 100% of the C nucleotide species given the concentration ratio. Those of ordinary skill in the art will appreciate that C. jejuni is an AT rich genome generally meaning that there is a higher incidence of A and T nucleotide species in the genome composition than C and G nucleotide species. Thus, the relative percentages for TA and GC concentrations illustrated in FIG. 4 reflect that fact and typically would be different for a GC rich genome (i.e. the genome of T. thermophilus).

The average completion efficiency (i.e. to estimate incomplete extension) and carry forward values of each individual nucleotide species were inferred using a method described in detail below. The last column presents the average read lengths (RL) of the high quality reads obtained where increased read length correlates to an increased level of optimization of the combination of each nucleotide species concentration that is balanced for the species specific incorporation efficiency of polymerase to degradation efficiency of apyrase.

TABLE 4
Effect of Nucleotide Concentration on Completion Efficiency/Carry Forward Values

| | relative | | | | | | | | | | |
|---|---|---|---|---|---|---|---|---|---|---|---|
| | concentration | | average completion efficiency | | | | average carry forward | | | | |
| Run | TA | CG | T | A | C | G | T | A | C | G | RL |
| 1 | 120% | 140% | 0.9968 | 0.9977 | 0.9988 | 0.9990 | 0.0006 | 0.0035 | 0.0062 | 0.0048 | 530 |
| 2 | 140% | 140% | 0.9975 | 0.9971 | 0.9986 | 0.9992 | 0.0013 | 0.0033 | 0.0058 | 0.0075 | 498 |
| 3 | 120% | 160% | 0.9959 | 0.9978 | 0.9991 | 0.9994 | 0.0008 | 0.0043 | 0.0065 | 0.0057 | 472 |

As will be apparent to those of ordinary skill, Table 4 illustrates that increasing the concentration of a particular pair of nucleotide species increases the corresponding completion efficiency for each species, but in most cases also increases the corresponding carry forward value for that species (the carry forward value for G nucleotide species did not follow this trend). The inferred values of completion efficiency and carry forward values are representative of the combined enzymatic activity which is directly related to the efficiency of polymerase and apyrase, respectively, towards each nucleotide species. An optimal set of nucleotide species concentration maximizes the completion efficiency and minimizes the carry forward values of each nucleotide species, resulting in the longest average read length which is a measure of sequencing performance. It will also be appreciated that the species specific differences become more significant as the number of flow cycles increases where every flow cycle increases the probability that some type of CAFIE error occurs.

The average completion efficiencies and carry forward values of the individual nucleotide species were inferred using nucleotide-specific CAFIE (carry forward and incompletion extension) fit. For example, the sequencing process produces raw sequence reads typically processed by an embodiment of Image and Signal Processing software such as application 135. In the present example, application 135 maps (also referred to as an alignment by those of ordinary skill in the art) the raw sequence reads generated to a reference genome sequence associated with the samples sequenced, such as a consensus reference sequence of C. jejuni. As will be evident to those of ordinary skill, mapping/aligning the raw uncorrected sequence reads to a reference sequence can be employed to determine where CAFIE error occurs and determination of incidence of nucleotide species specific CAFIE error.

In the presently described example application 135 mapped 200 uncorrected sequence reads, represented as flowgrams, to the reference genome sequence and the first 100 iterations of the nucleotide flows considered for determination of optimal concentration of each of the nucleotide species by inferring the completion efficiencies and carry forward values. The detected signal strengths of each of the four nucleotide species were first normalized using the median 1-mer value for all nucleotide species. The term “1-mer value” as used herein generally refers to a value reflecting the degree of detected signal in response to an incorporation of a nucleotide species at a single sequence position which should be substantially the same for each of the nucleotide species. Typically this the 1-mer value is easiest to calculate from detected signals obtained at the beginning of a sequencing run of a template nucleic acid molecule because the key element in an adaptor includes a single representative of each nucleotide species in a key element at a known position (i.e. a TCAG key element) and thus it is expected that only a single nucleotide of each species is incorporated at each of the four sequence positions.

The completion efficiency and carry forward values illustrated in Table 4 can be inferred using a three-stage minimization process for each individual sequence read. In the first stage, the following expression can be minimized to obtain the nucleotide species independent completion efficiency and carry forward value:

$$arg min {\sum }_{i} {\vert {M}^{- 1} ( v , λ , ɛ ) ( q - n ) \vert}^{2} for flow i that υ ( i ) = 0$$

where q is an uncorrected flowgram, v is an incorporation list derived from the reference flowgram (which is derived from the reference sequence of the read), λ is a nucleotide species independent completion efficiency parameter, ε is a nucleotide species independent carry forward value, M is a CAFIE matrix and n is the average noise. The incorporation list v is a vector of the length of the flowgram, which is 400 flows in the present example. The v(i) equals 1 if the reference flowgram shows one or more incorporations at flow i, and equals 0 if the reference flowgram shows no incorporation. This step seeks the set of parameters (λ, ε, n) that minimizes the square deviation of the negative flows (i.e. where the is no nucleotide species incorporation) from the mean noise. The minimization is performed using an initial guess values of (λ, ε)=(0.998, 0.005) and the initial guess values of n is set to the average normalized intensity of the first five negative flows (i.e. flows that v(i)=0). The search is performed with the lower bound (λ, ε, n)=(0.99, 0, 0) and the upper bound (1, 0.05, max(q(i)) where v(i)=0).

The second stage is to infer the nucleotide-specific completion efficiency and carry forward values and the average noise. The following expression is minimized:

$$arg min {\sum }_{i} {\vert {M}^{- 1} ( v , {λ}_{T} , {λ}_{A} , {λ}_{C} , {λ}_{G} , {ɛ}_{T} , {ɛ}_{A} , {ɛ}_{C} , {ɛ}_{G} ) ( q - n ) \vert}^{2}$$

$$for flow i that v ( i ) = 0$$

The search starts with the initial guess values (λ_{T}, λ_{A}, λ_{C}, λ_{G}, ε_{T}, ε_{A}, ε_{C}, ε_{G}, n)=(λ₁, λ₁, λ₁, λ₁, ε₁, ε₁, ε₁, ε₁, n₁) where λ₁, ε₁ and n₁ are the completion efficiency, carry forward and the average noise values obtained from the first stage. The lower and upper bounds of the search are (0.99, 0.99, 0.99, 0.99, 0, 0, 0, 0, 0) and (1, 1, 1, 1, 0.05, 0.05, 0.05, 0.05, max(q(i)) where v(i)=0).

The third and final stage is to infer the nucleotide-specific completion efficiency and carry forward values, the average noise and the phase shift (i.e. an incomplete extension of carry forward change in phasic synchrony). The following expression is minimized:

$$arg min {\sum }_{i} {\vert {M}^{- 1} ( v , {λ}_{T} , {λ}_{A} , {λ}_{C} , {λ}_{G} , {ɛ}_{T} , {ɛ}_{A} , {ɛ}_{C} , {ɛ}_{G} , φ ) ( q - n ) \vert}^{2}$$

$$for flow i that v ( i ) = 0 ,$$

where φ is the phase shift (i.e. phasic synchrony error from CAFIE effects). The search starts with the initial guess values from the first stage (λ_{T}, λ_{A}, λ_{C}, λ_{G}, ε_{T}, ε_{A}, ε_{C}, ε_{G}, n, φ)=(λ_{T2}, λ_{A2}, λ_{C2}, λ_{G2}, ε_{T2}, ε_{A2}, ε_{C2}, ε_{G2}, n₂, 0) where λ_{T2}, λ_{A2}, λ_{C2}, λ_{G2} are nucleotide-specific completion efficiency values obtained in the second stage, ε_{T2}, ε_{A2}, ε_{C2}, ε_{G2} are nucleotide specific carry forward values obtained in the second stage, and n₂ is the noise value obtained in the second stage. The lower and upper bounds of the search are (0.99, 0.99, 0.99, 0.99, 0, 0, 0, 0, 0, 0) and (1, 1, 1, 1, 0.05, 0.05, 0.05, 0.05, max(q(i)) where v(i)=0, 1) respectively.

The nucleotide specific completion efficiency and carry forward values listed in the Table 4 above are the sets of values obtained from the third stage calculations, averaging over 200 samples. By adjusting the nucleotide species concentration according to the trend of change of the completion efficiencies and carry forward values, the optimal set of nucleotide species concentrations that maximizes the completion efficiencies and minimizes the carry forward to the optimal level permitted by both the polymerase and apyrase enzymes and the sequencing condition can be obtained. For example, a titration curve may be plotted for nucleotide species concentration versus CAFIE effects that graphically illustrates the optimal nucleotide species concentration for the reaction environment parameters that includes the species specific enzyme activity levels.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

What is claimed is:

1. A method for optimizing sequencing performance, comprising the steps of:
a) calculating a nucleotide species specific degradation rate of an apyrase enzyme for a plurality of nucleotide species;
b) determining a concentration for each of the nucleotide species using the nucleotide species specific degradation rate;
c) iteratively providing the concentration of each of the nucleotide species in a reaction environment comprising a polymerase enzyme and a species of template nucleic acid molecule, wherein one or more molecules of the nucleotide species are incorporated into a nascent molecule in a sequencing reaction and the apyrase enzyme is introduced to the reaction environment to degrade unincorporated nucleotide species molecules; and
d) detecting a signal in response to the incorporation of the nucleotide species.

2. The method of claim 1, wherein:
the concentration for each of the nucleotide species is determined to provide a balance of the species specific degradation rate relative to an incorporation efficiency of a polymerase enzyme.

3. The method of claim 1, wherein:
the nucleotide species specific degradation rate is calculated using the Michaelis-Menten equation.

4. The method of claim 1, wherein:
the nucleotide species comprise dTTP, α-thio-dATP, dCTP and dGTP.

5. The method of claim 1, wherein:
the nucleotide species comprise dTTP, dATP, dCTP and dGTP.

6. The method of claim 1, wherein:
the concentration for each of the nucleotide species is normalized to a dCTP species that comprises the lowest concentration and activity values among the nucleotide species.

7. The method of claim 1, wherein:
the concentrations for a plurality of the nucleotide species are different from one another.

8. The method of claim 1, wherein:
the concentrations for each of the nucleotide species are different from one another.

9. The method of claim 2, wherein:
the balance comprises optimizing the incorporation efficiency by maintaining the concentration of each of the nucleotide species in a reaction environment for sufficient time for incorporation to occur prior to degradation by the apyrase enzyme.

10. The method of claim 1, wherein:
the molecules of the nucleotide species are incorporated into the nascent molecule at one or more positions based upon complementarity of the nucleotide species to the nucleic acid template species.

11. The method of claim 1, further comprising:
e) generating a sequence read from the detected signals, where the sequence read comprises a sequence composition of the species of template nucleic acid molecule.

12. A method for optimizing sequencing performance, comprising the steps of:
a) introducing a plurality of relative concentrations of nucleotide species into a type of reaction environment comprising a polymerase enzyme, an apyrase enzyme, and a species of a template nucleic acid molecule to produce an uncorrected sequence composition of the species of template nucleic acid molecule;
b) determining a completion efficiency value and a carry forward value for each of the nucleotide species from the uncorrected sequence composition and a reference sequence composition of the species of template nucleic acid molecule; and
c) identifying a species specific concentration for each of a plurality of nucleic acid species using the nucleotide species specific completion efficiency value and the nucleotide species specific carry forward value, wherein the species specific concentrations are optimized to minimize error produced by a sequencing reaction in the type of reaction environment.

13. The method of claim 12, further comprising:
d) executing the sequencing reaction using the type of reaction environment, wherein the sequencing reaction comprises the steps of:
i. iteratively delivering each of the nucleic acid species at the species specific concentration to the type of reaction environment comprising a second species of template nucleic acid molecule and the polymerase enzyme, wherein the apyrase enzyme is delivered to the reaction environment between iterations of delivery of the nucleic acid species; and
ii. detecting a plurality of signals generated in response to incorporation of the nucleic acid species by the polymerase.

14. The method of claim 13, wherein:
the apyrase is delivered to the reaction environment at a nucleotide species specific concentration.

15. The method of claim 12, wherein:
the type of reaction environment comprises a well disposed on a planar substrate, wherein the planar substrate comprises a plurality of wells.

16. The method of claim 12, wherein:
the relative concentrations comprise a first percentage of an A nucleotide species concentration and a T nucleotide species concentration relative to a second percentage of a G nucleotide species concentration and a C nucleotide species concentration.

17. The method of claim 16, wherein:
the first percentage and the second percentage are defined according to the sequence composition of the species of template nucleic acid molecule, wherein the sequence composition is AT rich or GC rich.

18. The method of claim 12, wherein:
the nucleotide species comprise dTTP, α-thio-dATP, dCTP and dGTP.

19. The method of claim 12, wherein:
the nucleotide species comprise dTTP, dATP, dCTP and dGTP.

20. A system for optimizing sequencing performance, comprising:
a) a computer comprising executable code stored thereon wherein the executable code performs a method comprising the steps of:
i. calculating a nucleotide species specific degradation rate of an apyrase enzyme for a plurality of nucleotide species;
ii. determining a concentration for each of the nucleotide species using the nucleotide species specific degradation rate, wherein the concentration for each of the nucleotide species is determined to provide a balance of the species specific degradation efficiency relative to an incorporation efficiency of a polymerase enzyme; and
b) a sequencing instrument that performs a method comprising the steps of:
i. iteratively providing the concentration of each of the nucleotide species in a reaction environment comprising a polymerase enzyme and a species of template nucleic acid molecule, wherein one or more molecules of the nucleotide species are incorporated into a nascent molecule in a sequencing reaction and the apyrase enzyme is introduced to the reaction environment to degrade unincorporated nucleotide species molecules; and
ii. detecting a signal in response to the incorporation of the nucleotide species.
